(19) United States
(12) Patent Application Publication
(10) Pub. No.: US 2008/0125493 A1
(43) Pub. Date: May 29, 2008

(54) Use of agomelatine in obtaining medicaments intended for the treatment of periventricular leukomalacia

(75) Inventors: Michael Spedding, Le Vesinet (FR); Elisabeth Mocaer, Neuilly Sur Seine (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, COURBEVOIE CEDEX (FR)

(21) Appl. No.: 11/986,011

(22) Filed: Nov. 19, 2007

(30) Foreign Application Priority Data
Nov. 24, 2006 (FR) 06.10294

(51) Int. Cl. A61K 31/165; A61P 25/00

(52) U.S. Cl. 514/630

(57) ABSTRACT

The present invention relates to the use of agomelatine, or N-[2-(7-methoxy-1-naphthyl)ethyl]acetamide, in obtaining medicaments intended for the treatment of periventricular leukomalacia.

The present invention relates to the use of agomelatine, or N-[2-(7-methoxy-1-naphthyl)ethyl]acetamide of formula (I):

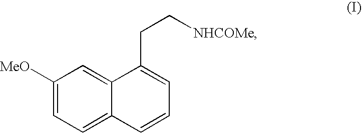

and also its hydrates, crystalline forms and addition salts with a pharmaceutically acceptable acid or base, in obtaining medicaments intended for the treatment of periventricular leukomalacia.

Agomelatine, or N-[2-(7-methoxy-1-naphthyl)ethyl]acetamide, has the double characteristic of being, on the one hand, an agonist of receptors of the melatoninergic system and, on the other hand, an antagonist of the 5-HT_{2C} receptor. These properties provide it with activity in the central nervous system and, more especially, in the treatment of major depression, seasonal affective disorder, sleep disorders, cardiovascular pathologies, pathologies of the digestive system, insomnia and fatigue due to jet-lag, appetite disorders and obesity.

Agomelatine, its preparation and its use in therapeutics have been described in European Patent Specifications EP 0 447 285 and EP 1 564 202.

The Applicant has now found that agomelatine or N-[2-(7-methoxy-1-naphthyl)ethyl]-acetamide—and also its hydrates, crystalline forms and addition salts with a pharmaceutically acceptable acid or base—has valuable properties allowing its use in the treatment of periventricular leukomalacia.

Periventricular leukomalacia is the most frequent cause of cerebral palsy in premature infants. This early neonatal disorder is due to the formation of single or multiple lesions of the ring of periventricular white matter, occurring during prenatal or neonatal life, between 20 and 34 weeks post-conception, exceptionally even up until term. Periventricular leukomalacia is responsible for the majority of motor sequelae of prematurity. Nevertheless, other neurological sequelae or other complications, although less frequent, may be observed, indicating extension of the lesions beyond the periventricular white matter: growth anomalies of the cranial perimeter, essentially indicating anomalies of the proliferation and/or regression of dendrites; intellectual deficit secondary to an associated cortical insult; specific disorders of development affecting one out of two or three children at the age of entering the ‘cours préparatoire’ (i.e. when starting school at the age of seven); sensory deficits, albeit exceptional, secondary to insults of the auditory or visual radiations; an increased frequency of sudden infant death. Added to the suffering of parents confronted with the problem of neurological sequelae or with the death of their child is the helplessness of a medical team which is completely at a loss when faced with the appearance of extensive periventricular leukomalacia in neonates who sometimes have no other complications of prematurity: in fact there is currently no therapeutic strategy that makes it possible to prevent or to limit the extension of such lesions. Furthermore, the increase in the frequency of multiple pregnancies and also the ever further lowering of the limits of viability of highly premature infants are resulting in a recognised increase in the incidence of periventricular leukomalacia, which is the principal challenge facing neonatalogists. The causes of the disorder have recently been established as being multi-factorial: pre-conception, prenatal and perinatal factors may be involved in the formation of lesions during development of the brain. Among those factors there may be mentioned episodes of hypoxia-ischaemia, endocrine imbalances, genetic factors, disorders related to growth factors, maternal infections resulting in excess cytokine production, exposure to pro-inflammatory agents, etc. These multiple risk factors have common molecular manifestations, especially excess release of excitatory amino acids and increased production of reactive oxygenated species.

The Applicant has now found that agomelatine has a neuroprotective effect which has the effect of promoting repair mechanisms of secondary lesions of the periventricular white matter. Agomelatine accordingly constitutes a new approach to the treatment of periventricular leukomalacia. In addition, agomelatine has the characteristic of being very well tolerated and of not having problems of drug interactions, making it a treatment that is especially suitable in this indication.

The invention accordingly relates to the use of agomelatine, and also its hydrates, crystalline forms and addition salts with a pharmaceutically acceptable acid or base, in obtaining pharmaceutical compositions intended for the treatment of periventricular leukomalacia.

The invention relates especially to the use of agomelatine obtained in crystalline form II, described in Patent Application EP 1 564 202, in obtaining pharmaceutical compositions intended for the treatment of periventricular leukomalacia.

The pharmaceutical compositions will be presented in forms suitable for administration by the oral, parenteral, transcutaneous, nasal, rectal or perlingual route, and especially in the form of injectable preparations, tablets, sublingual tablets, glossettes, gelatin capsules, capsules, lozenges, suppositories, creams, ointments, dermal gels etc.

Besides agomelatine, the pharmaceutical compositions according to the invention comprise one or more excipients or carriers selected from diluents, lubricants, binders, disintegration agents, absorbents, colourants, sweeteners etc.

By way of non-limiting example there may be mentioned:
as diluents: lactose, dextrose, sucrose, mannitol, sorbitol, cellulose, glycerol,
as lubricants: silica, talc, stearic acid and its magnesium and calcium salts, polyethylene glycol,
as binders: aluminium and magnesium silicate, starch, gelatin, tragacanth, methylcellulose, sodium carboxymethylcellulose and polyvinylpyrrolidone,
as disintegrants: agar, alginic acid and its sodium salt, effervescent mixtures.

The useful dosage varies according to the age and weight of the patient, the administration route, the nature of the disorder and any associated treatments and ranges from 1 mg to 50 mg of agomelatine per 24 hours.

Preferably, the daily dose of agomelatine will be 25 mg per day, with the possibility of increasing to 50 mg per day.

Pharmaceutical Composition:

Formula for the preparation of 1000 tablets each containing 25 mg of active ingredient:

| | |
|---|---|
| N-[2-(7-methoxy-1-naphthyl)ethyl]acetamide | 25 g |
| Lactose monohydrate | 62 g |
| Magnesium stearate | 1.3 g |
| Povidone | 9 g |
| Anhydrous colloidal silica | 0.3 g |
| Cellulose sodium glycolate | 30 g |
| Stearic acid | 2.6 g |

Pharmacological Study

The neuroprotective effects of agomelatine were observed in 5-day-old baby mice in which lesions of the white matter of the brain were produced by intracerebral injection of ibotenate. Immediately after the administration of 10 μg of ibotenate, there are injected by the i.p. route, in a final volume of 5 μl, from 0.005 to 5 mg/kg of agomelatine, 10 mg/kg of fluoxetine used as standard antidepressant control, or the solvent alone. In a second experiment, the i.p. injection following the intracerebral injection of ibotenate is carried out 2 hours, 4 hours or 8 hours later.

Results: After the administration of ibotenate, the infant mice develop cortical lesions and also lesions in the periventricular white matter. Co-administration of agomelatine is reflected by dose-dependent reduction of the lesions of the white matter reaching 59% reduction for administration of 5 mg/kg. No significant effect on the lesions of the white matter is observed in the case of co-administration of fluoxetine or of solvent alone (PBS: “Phosphate Buffered Saline”).

The neuroprotection observed with agomelatine decreases when the administration is carried out between 0 and 4 hours after the injection of ibotenate and then significant neuroprotection is restored when the administration is carried out 8 hours after the injection of ibotenate. The results are plotted below:

The animals administered ibotenate develop lesions whose size increases for the first 24 hours following the injection and then stabilises. In the animals co-treated with agomelatine (concomitantly or after a period of 8 hours) the same lesions are observed for the first 24 hours, followed by very major regression over the next 4 days.

1- A method for treating a living animal body, including a human, afflicted with periventricular leukomalacia comprising the step of administering to the living animal body, including a human, an amount of agomelatine, or N-[2-(7-methoxy-1-naphthyl)ethyl]acetamide, or a hydrate, crystalline form or addition salt thereof with a pharmaceutically acceptable acid or base, which is effective for treatment of periventricular leukomalacia.

2- The method of claim 1, wherein the agomelatine is in crystalline form II.

3- A pharmaceutical composition comprising agomelatine, or a hydrate, crystalline form or addition salt thereof with a pharmaceutically acceptable acid or base, alone or in combination with one or more pharmaceutically acceptable excipients.

4- The pharmaceutical composition of claim 3, wherein the agomelatine is in crystalline form II.
